(19) United States
(12) Patent Application Publication
(10) Pub. No.: US 2011/0022075 A1
(43) Pub. Date: Jan. 27, 2011

(54) Aspiration Catheter

(75) Inventors: Frank Christiansen, Haslev (DK); Per Elgård, Haslev (DK); Anders Ginge Jensen, Ringsted (DK); Kian Olsen, Vallensbaek Strand (DK); Carsten Skødt, Sorø (DK)

Correspondence Address: COOK GROUP PATENT OFFICE, P.O. BOX 2269, BLOOMINGTON, IN, 47402, US

(73) Assignees: William Cock Europe ApS, Bjaeverskov (DK); Cook Incorporated, Bloomington, IN (US)

(21) Appl. No.: 12/841,799

(22) Filed: Jul. 22, 2010

(30) Foreign Application Priority Data
Jul. 22, 2009 (GB) 0912775.4

(51) Int. Cl. A61B 17/22; A61M 25/00

(52) U.S. Cl. 606/200; 604/524

(57) ABSTRACT

To prevent a small diameter catheter 10 of soft material from collapsing when being used to aspirate a blood clot 25, the lumen 16 of the catheter contains a longitudinal member 26 having radially-extending fin elements 33. The longitudinal member may itself be a catheter having a lumen 22 through which passes a wire 27 having at its distal end an expandable basket 28 for retrieving the clot.

### TECHNICAL FIELD

The present invention relates to an aspiration catheter and to a deployment arrangement with an aspiration device. In particular it relates to a small diameter catheter suitable for aspirating a blood clot or thrombus in cerebral arteries.

### BACKGROUND OF THE INVENTION

Devices for occluding the vasculature of the human body are known which have a relatively small cross-section during delivery and a larger cross-section in their deployed configuration. Such a vaso-occlusive coil is disclosed in U.S. Pat. No. 6,638,291. A catch-type clot retriever is also known, in which a basket is delivered with a small cross-section past the clot, deployed by expanding it, and then retrieved to catch the clot.

There is also known a Merci (Trade Mark) L-series clot retriever comprising a tapering helical coil. This is moved past the clot in a straight configuration; a surrounding sheath is then retracted which allows the coil to expand to its helical configuration.

U.S. Pat. No. 4,954,129 discloses a process for flushing clots from an intravascular probe. A catheter surrounds a probe having ribs which serve to centre the probe with respect to the catheter.

Because cerebral blood vessels have small diameters, it would be helpful to surgeons to have available a clot retrieval arrangement which does not include a component which is expanded after delivery, or at least an arrangement in which such a component is optional.

### SUMMARY OF THE INVENTION

Aspects of the present invention seek to overcome or reduce one or more of the above problems.

According to a first aspect of the present invention, there is provided an intravascular aspiration arrangement comprising a collapsible catheter, the catheter having a lumen with a longitudinal member extending therethrough, said longitudinal member being spaced from the internal wall of the catheter by a plurality of radially-projecting fin elements.

An advantage of this arrangement is that the diameter and wall thickness of the catheter can be small enough for it to reach a distant cerebral thrombus, yet it is prevented from collapsing, thus allowing aspiration through the deployed catheter.

Preferably, the fin elements are located on the longitudinal member. This provides an arrangement which is easy to manufacture. In addition if the fin elements are on the inside wall of the catheter, there are two potential problems:

(i) the longitudinal member may slip radially between the fin elements thus allowing the catheter to collapse to restrict aspiration;
(ii) if such an arrangement is used with a basket or other retrieval element at the distal end of the longitudinal member, the passage of the element through the catheter might be hindered or completely prevented.

At least three fin elements and preferably four fin elements are provided. Such arrangements ensure that the catheter is kept open. More than four fin elements may be provided if desired, but with numbers higher than six, the free cross-sectional area within the catheter becomes reduced. In preferred embodiments, at least some, preferably the majority, and most preferably all of the fin elements extend along substantially the entire length of the catheter.

In some embodiments, a distal end portion of the catheter is narrowed compared to the remainder of the catheter. Here, the fin elements preferably extend along substantially all of the narrowed portion.

The fin elements may be intermittent rather than continuous along their length.

In preferred embodiments, the longitudinal member is in the form of a second catheter having a lumen through which passes a wire having an expandable retrieval member at a distal end thereof. Although this may slightly increase the dimensions of the arrangement, and in particular its diameter, such an arrangement has the advantage of catching any parts of the clot released during aspiration and of retrieving any parts of the clot remaining after aspiration. Thus the aspiration process removes the soft, relatively fluid parts of the thrombus and the retrieval member, e.g. a basket, removes the relatively hard and/or larger parts.

In preferred embodiments the first catheter is in two parts with a distal part being of a softer and/or more flexible material than a proximal part. The stiffer proximal part enables the catheter to be pushed through the vascular system of the patient to the location of the clot. The pliant nature of the distal part enables it to enter cranial arteries of small diameter. The distal and proximal parts may be interconnected by a frictional grip connection.

According to a second aspect of the present invention, there is provided an intravascular deployment arrangement comprising a collapsible catheter having a lumen with a longitudinal member passing therethrough, a handle at a proximal end of the catheter and having means for advancing the longitudinal member to a distal end of the catheter, and suction means for aspirating the lumen of the catheter, wherein the longitudinal member is spaced from the wall of the lumen of the catheter by a plurality of radially-projecting fin elements.

The handle may include means for advancing the catheter through the vasculature of a patient. The advancing means preferably comprises means for advancing a guide wire through the vasculature of a patient and means for advancing the catheter over the deployed guide wire.

### BRIEF DESCRIPTION OF THE DRAWING

Preferred embodiments of the present invention will now be described, by way of example only, with reference to the accompanying drawings, of which:

FIG. 1 shows part of an aspiration catheter of an aspiration arrangement in accordance with a first embodiment of the present invention;

FIG. 2 shows a sectional view of an aspiration arrangement in accordance with the first embodiment inserted in a patient's vessel;

FIG. 3 is an enlarged cross-section of the catheter on the line A-A;

FIG. 4 is a cross-section of the catheter of an aspiration arrangement in accordance with a second embodiment of the present invention;

FIG. 5 shows part of a modified aspiration arrangement before deployment of a retrieval member thereof; and

FIG. 6 is a view corresponding to FIG. 5 after deployment of the retrieval member.

### DETAILED DESCRIPTION

It would be very useful to surgeons to be able to improve the success rate for the difficult task of removing soft thrombosis from intracranial arteries. Until now, it has not been possible to obtain aspiration catheters for a distant cerebral thrombus. Such catheters need to be narrow enough and flexible enough to be deployed in the small cerebral arteries while being stiff enough to avoid collapse during aspiration.

Part of a catheter 10 intended to contribute to achieving the above object is shown in FIG. 1. The catheter comprises a first, proximal part 11 having a first diameter d₁ and a second, distal part 12 having a second diameter d₂ substantially smaller than the first diameter. FIG. 1 shows the region 15 where the two parts 11, 12 are connected together. The catheter has a lumen 16.

In the present specification, the term proximal is used for a part of the aspirator arrangement which is nearer to an operator of the arrangement. The term distal is used for a part of the arrangement which is further from an operator.

Parts 11 and 12 are formed of a synthetic material such as nylon, with the material of part 12 being significantly softer than that of part 11.

The first part 11 may be formed by a cable tube and have a diameter of 4 to 8 French, preferably 6 French. Apart from in its tapering section 17, the second part 12 has a diameter of 2 to 4 French, preferably 3 French. The second part may be formed by a microcatheter, e.g. a MiraFlex (Trade Mark) catheter. The proximal end of part 12 overlaps the distal end of part 11 to effect a firm grip which provides a secure frictional connection between the two parts. The diameter of the non-tapered section of the second part is approximately one half of the diameter of the first part 11.

Part 11 typically extends from an introducer device outside the patient's body to a location near the clot, and thus has a length of between 1.25 m and 1.50 m. Part 12 has a length of between 6 mm and 8 mm. Tapering section 17 has a length of between 1 mm and 2 mm.

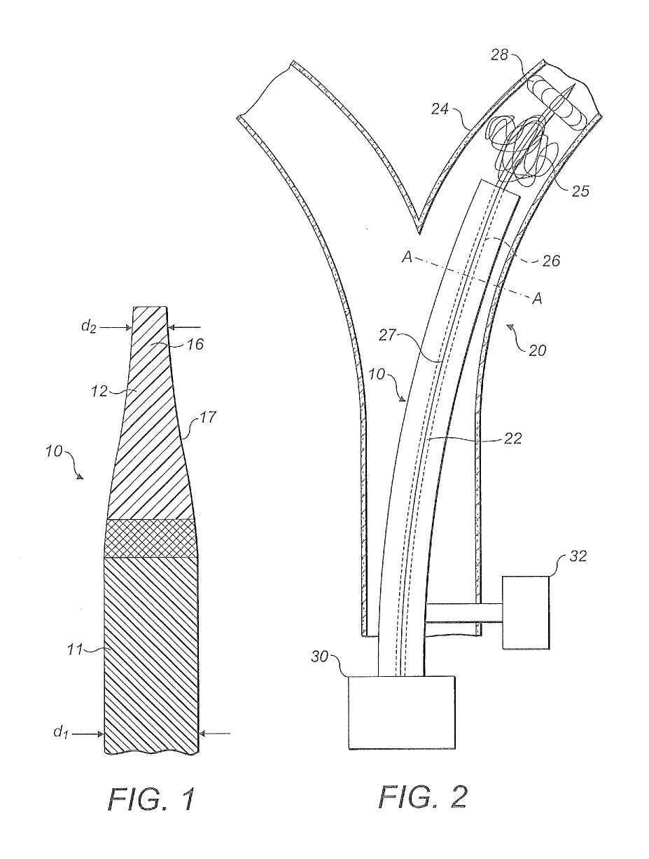

FIG. 2 shows an aspiration arrangement 20 in which the catheter 10 of FIG. 1 is used to remove a soft thrombosis 25 from an intracranial artery 24. FIG. 2 shows the distal end of the second part 12 of the catheter in a position in which it has been moved up to a clot or thrombus 25. Extending from a handle (not shown) at the proximal end of an introducer device for the aspirator arrangement is a basket catheter 26. The introducer device is indicated schematically at 30. The basket catheter has a lumen 22 and is illustrated in FIG. 2 as having passed through the clot 25.

Also extending from the handle and inside the lumen 22 of catheter 26 is a wire 27 with a clot-retrieving basket 28 at the distal end thereof. When located within catheter 26, the basket 28 is constrained to a narrow elongated configuration within lumen 22. However, the wire 27 is illustrated in FIG. 2 as having its distal end pushed beyond the distal end of catheter 26. In view of its elastic nature the basket 28 is self-expanding and is shown expanded within the artery 24 and beyond the clot 25.

A source of suction 32 shown schematically in FIG. 2 is connected to the proximal end of catheter 10, preferably adjacent to the handle of the introducer device 30.

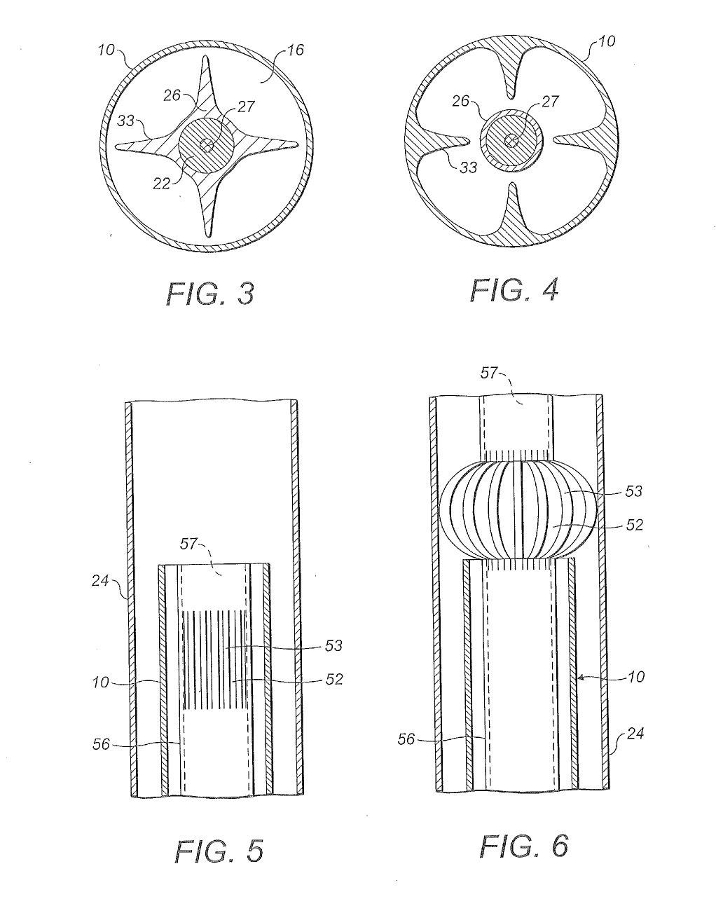

There is shown in FIG. 3 a cross-section of the catheter 10 along the line A-A of FIG. 2. Basket catheter 26 is shown extending along lumen 16 of catheter 10, and wire 27 is shown extending along lumen 22 of basket catheter 26. The external wall of basket catheter 26 is provided with four equally-spaced fins 33 which extend across most of the radial spacing between the catheter 26 and the internal wall of catheter 10.

A preferred method of deployment and use of the aspiration arrangement will now be described.

A guide wire (not shown) is first deployed in the usual manner to the location of the clot 25. The catheter 10 is then advanced over the guide wire to its location as shown in FIG. 2. Because of its relatively small diameter, at least the distal part 12 of the catheter passes relatively easily through the small cranial vessels. The guide wire is then removed. Using the introducer device 30, the basket catheter 26 with the wire 27 therein is then inserted into catheter 10 and advanced until it has passed through the clot 25. In view of its small diameter, the catheter 26 passes relatively easily through the clot 25. Basket 28 remains in its collapsed configuration within catheter 26. Using the introducer device 30, the wire 27 with the basket 28 therein is advanced beyond the end of catheter 26, so that the basket expands to its configuration as shown in FIG. 2.

The source of suction 32 is then switched on and at least the soft parts of clot 25 are aspirated out of the patient along catheter 10. The tendency of the walls of catheter 10 to collapse is resisted by fins 33 so that lumen 16 remains substantially free for the passage of parts of the clot. As aspiration proceeds, the basket 28 serves to catch any parts of the clot which become detached. When aspiration is completed, the entire aspiration arrangement is withdrawn from the patient, the basket 28 serving to entrain any remaining parts of the clot, e.g. any parts of the clot which are too hard and/or too large to have been removed by aspiration.

The above-described arrangement has several advantages. It provides the surgeon with a technique to remove clots from blood-vessels which are too small to be entered by existing clot-removal devices. The fins 33 ensure that the lumen 16 is maintained open at all times during aspiration. The construction of catheter 10 in two parts 11 and 12 combines the advantages of the pushability of part 11, so that it can be directed to the desired location, with properties of part 12 of size and degree of softness, which allow it to enter small blood vessels.

Numerous modifications can be made to the above-described arrangement. For example, the catheter 26 can have three radial fins 33 instead of four. More than four radial fins may be provided, but more than six would leave only a restricted space for the passage of clot material. The fins 33 may be of any desired size and shape. They preferably extend more than 50% of the radial distance from catheter 26 to the inside of catheter 10, more preferably more than 75% of the distance and most preferably substantially 90% of the distance. They can extend more than 90% of the radial distance, but this makes advance of the catheter 26 along lumen 16 more difficult.

Each fin element is preferably continuous along its entire length. Alternatively, there may be short gaps along its length.

Instead of an arrangement in which the basket 28 is self-expanding, separate means may be provided for expanding the basket. A disadvantage of this modification, however, is that a separate actuator undesirably increases the bulk of the arrangement.

In another modification, the basket 28 is of a shape memory material such as Nitinol.

As shown in the modification of FIG. 4, the fin elements 33 can be arranged on the inside wall of catheter 10. However, there is an increased risk here of at least partial collapse of the soft material of catheter 10 caused by catheter 26 passing between two adjacent fin elements 33.

The fin elements have any desired shape and thickness. Thin fin elements are preferred since they permit maximum aspiration flow.

Instead of the tapering section 17 being provided in distal part 12, the proximal part 11 may incorporate a tapering section. Alternatively the two parts 11 and 12 may be integral parts of the same catheter. In addition the taper may be omitted so that the catheter has a uniform diameter throughout its length, but this makes it more difficult to advance the catheter through narrow body vessels.

Clot retrieval basket 28 can be omitted. An advantage of this modification is that catheter 26 does not need to accommodate the basket even in its collapsed configuration. Accordingly wire 27 can be omitted and the lumen 22 of the catheter 26 can be much smaller. Indeed the lumen may be omitted so that the catheter 26 is itself replaced by a wire or other solid member.

FIGS. 5 and 6 show a modification in which the retrieval device 55 is formed by a section of a catheter 56 surrounding a wire 57. At or adjacent their distal ends, the catheter 56 and wire 57 are fixedly attached to each other. In the region where the device 55 is to be formed, the wall of the catheter has a plurality of longitudinal slits 52 defining intermediate strands 53.

FIG. 5 shows the arrangement as it is inserted along catheter 10. FIG. 6 shows the deployment of the retrieval device 55 after it has emerged from the distal end of catheter 10. Since the distal ends of catheter 56 and wire 57 are fixed to each other, a push on catheter 56 in the distal direction relative to wire 57 causes the strands 53 to flex in the manner of a Chinese lantern and thus slits 52 open up to form an expanded retrieval device. Instead of pushing on catheter 56, the same result can be obtained by pulling on wire 57. In either case, appropriate pushing or pulling means and retention means are provided at the proximal end at introducer 30.

What is claimed is:

1. An intravascular aspiration arrangement comprising a catheter collapsible when subjected to aspiration, the catheter having a lumen with a longitudinal member extending therethrough, said longitudinal member being spaced from the internal wall of the catheter by a plurality of radially-projecting fin elements, the catheter comprising a distal part and a proximal part, the distal and proximal parts being interconnected and the distal part being of a softer and/or more flexible material than the proximal part.

2. An arrangement according to claim 1, wherein the fin elements are located on the longitudinal member.

3. An arrangement according to claim 2 comprising three to six fin is elements.

4. An arrangement according to claim 3 comprising four fin elements.

5. An arrangement according to claim 1, wherein the fin elements extend along substantially the entire length of the catheter.

6. An arrangement according to claim 1, wherein the catheter has a tapered portion whereby a distal part of the catheter has a smaller diameter than a proximal part of the catheter.

7. An arrangement according to claim 6, wherein the distal part is connected to the proximal part by a frictional grip connection.

8. An arrangement according to claim 6, wherein the distal part is of a softer and/or more flexible material than the proximal part.

9. An arrangement according to claim 6, wherein the fin elements extend along substantially the entire length of the distal part of the catheter.

10. An arrangement according to claim 1, wherein the longitudinal member is in the form of a second catheter having a lumen through which passes a wire having an expandable retrieval member at a distal end thereof.

11. An arrangement according to claim 1, wherein the longitudinal member is in the form of a second catheter having a lumen through which passes a wire, the walls of a distal portion of the second catheter being deformable to form a retrieval member.

12. An intravascular deployment arrangement comprising a collapsible catheter having a lumen with a longitudinal member passing therethrough, a handle at a proximal end of the catheter and having means for advancing the longitudinal member to a distal end of the catheter, and suction means for aspirating the lumen of the catheter, wherein the longitudinal member is spaced from the wall of the lumen of the catheter by a plurality of radially-projecting fin elements.

13. An arrangement according to claim 12 wherein the longitudinal member is constituted by a second catheter having a second lumen and the arrangement further comprises a retrieval member arranged to pass through the second lumen, the handle having means for advancing the retrieval member beyond the distal end of the second catheter.

14. An arrangement according to claim 13, wherein the retrieval member comprises an expandable basket located at the distal end of a wire.

15. An arrangement according to claim 12 wherein the longitudinal member is in the form of a second catheter having a lumen through which passes a wire, the walls of a distal portion of the second catheter being deformable to form a retrieval member.

16. An arrangement according to claim 12, wherein the handle has means for advancing the catheter through the vasculature of a patient.

17. An arrangement according to claim 12, wherein the catheter has a tapered portion whereby a distal part of the catheter has a smaller diameter than a proximal part of the catheter.

18. An arrangement according to claim 17, wherein the distal part is connected to the proximal part by a frictional grip connection.

19. An arrangement according to claim 17, wherein the distal part is of a softer and/or more flexible material than the proximal part.

20. An arrangement according to claim 17, wherein the fin elements extend along substantially the entire length of the distal part of the catheter.
